(19) United States
(12) Patent Application Publication
(10) Pub. No.: US 2006/0276790 A1
(43) Pub. Date: Dec. 7, 2006

(54) Minimally invasive facet joint repair

(75) Inventors: John M. Dawson, Chaska, MN (US); Adam Shinbrot, St. Louis Park, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/213,105

(22) Filed: Aug. 26, 2005

Related U.S. Application Data
(60) Provisional application No. 60/686,771, filed on Jun. 2, 2005.

(51) Int. Cl. A61B 17/70; A61F 2/44

(52) U.S. Cl. 606/61; 623/17.11

(57) ABSTRACT

The present invention is an apparatus and method for stabilizing the facet joints of the spine. One aspect of the present invention is the stabilization of the facet joint by insertion of a facet implant through two opposing facet surfaces. The facet implant includes a proximal, distal and intermediate member. The distal member is inserted through the facet joint and into a facet surface. The intermediate member is then placed in the area between the facet surfaces. The proximal member then is brought closer to the distal member to deploy the intermediate member into the space between the facet surfaces. Once deployed, the intermediate member provides a cushioning or restoring force to the facet joint such as to stabilize movement of the joint.

### CROSS-REFERENCE TO RELATED APPLICATIONS

This application claims priority from U.S. Provisional Patent Application No. 60/686,771, filed on Jun. 2, 2005, entitled “Minimally Invasive Facet Joint Repair”, which is incorporated herein by reference in its entirety.

### TECHNICAL FIELD

The present invention is related to spinal stabilization devices. More particularly, the present invention relates to an apparatus and method for providing facet joint stabilization in the vertebrae while addressing back pain.

### BACKGROUND

The spinal column is a highly complex system of bones and connective tissues that provides support for the body and protects the delicate spinal cord and nerves. The spinal column includes a series of vertebrae stacked one on top of the other, each vertebral body including an inner or central portion of relatively weak cancellous bone and an outer portion of relatively strong cortical bone. Situated between each vertebral body is an intervertebral disc that cushions and dampens compressive forces experienced by the spinal column. A vertebral canal containing the spinal cord and nerves is located behind the vertebral bodies.

The bones and connective tissue of an adult human spinal column consists of more than 20 discrete bones coupled sequentially to one another by a tri-joint complex which consist of an anterior disc and two posterior facet joints. The anterior discs of adjacent bones are cushioned by cartilage spacers referred to as intervertebral discs. A few parts of the spine include the pedicle, the laminar arch, the facet, the spinous process, the transverse process, the vertical canal and the vertebral body. The vertebral body is the cylinder-shaped weight bearing-structure of the vertebra. The lamina are flat plates on the outer wall of the vertebral canal, which is formed between the vertebral body and the lamina and occupied by the spinal cord. The pedicle connects the lamina with the vertebral body. The spinous process protrudes from the back of the vertebra such that muscles and ligaments may attach thereto. Finally, the transverse process sticks out the sides of each vertebra and is another place where muscles and ligaments may attach to the spine.

A facet joint is a pair of opposing facets and the capsule around it. There are four facet surfaces associated with each vertebra: a pair of superior facets and a pair of inferior facets. The facet joints combine with the disc space to create a three-joint complex at each vertebral level. Generally, superior facets in the cervical region face upward; in the thoracic region, backward; in the lumbar region, medially. The change in orientation from cervical to thoracic is gradual, but from thoracic to lumbar, abrupt. The facet surfaces are covered with articular cartilage which allows the joints to glide smoothly (articulate) against each other. The facet joints (also called the zygapophyseal or apophyseal joints) are important because they interlock adjacent vertebrae, provide stability to the spine, and control its motion. Facet joints are synovial joints in that the connective tissue capsule produces a synovial fluid to nourish and lubricate the joint. The combination of the cartilage and the fluid allows the joint to move with little friction.

The facet joint is a bearing surface that moves in slip translation and include superior/inferior and medial/lateral degrees of freedom (DOF). Limited motion perpendicular to the articular surfaces (compression and distraction) and three limited rotations (flexion/extension, abduction/adduction, and internal external rotation) are also possible. Thus, the facet joint has six degrees of freedom. This is an important factor when considering treatment solutions that include facet fixation or repair. A successful treatment strategy will preserve as many of the degrees of freedom as possible.

Often the first treatment of the spine for facet pain includes facet blocks (injections). In addition, a surgical procedure in which spinal nerve roots are destroyed (rhizotomy) might be performed to relieve intractable pain. There are presently few other options for treating the facet joints of the spine.

### SUMMARY

This invention presents a unique, innovative solution for managing back pain. Whereas contemporary facet joint replacements are highly invasive and require a total facetectomy, this treatment paradigm preserves all or substantially all of the bones and the joint capsule. The implant could be placed percutaneously, perhaps in the same manner as a facet screw. The surgical technique therefore builds upon skills already possessed by the surgeon. In addition, the present invention could be used in conjunction with other technologies, say, nucleus replacement and total disc replacements. Because the present invention may employ minimally invasive or percutaneous methods it does not preclude later treatment by more invasive means.

One embodiment of the present invention is a facet implant for insertion into a facet joint including a distal member, a proximal member, and an intermediate member connecting the distal member and the proximal member, the intermediate member for positioning between a first facet surface and a second facet surface and transformable between a first shape and a second shape.

Another embodiment of the present invention is a method of preparing a facet joint for fusion including drilling a receiving hole through a first facet surface, inserting a facet tool into the receiving hole, the facet tool including a number of strips positioned between the opposing facet surfaces wherein the strips are transformable between a first shape and a second shape and wherein when the strips are in the second shape the strips exposing a cutting surface, and moving the facet tool such that the cutting strips abrade a desired amount of cartilage from the facet joint.

### BRIEF DESCRIPTION OF THE DRAWINGS

FIG. 1A is a side plan view of a facet implant of the present invention.

FIGS. 1B-E are side plan views of the facet implant of FIG. 1A in various states of deployment.

FIG. 2A is a perspective view of the facet implant of FIG. 1A.

FIGS. 2B-E are perspective views of the facet implant of FIG. 2A in various states of deployment.

FIG. 3A is a side plan view of an alternative embodiment of the facet implant of the present invention.

FIGS. 3B-E are side plan views of the facet implant of FIG. 3A in various states of deployment.

FIG. 4 is a perspective view of a facet implant of the present invention inserted into the facet joint of a spine.

FIG. 5 is another perspective view of a facet implant of the present invention inserted into the facet joint of a spine.

FIG. 6 is a side plan view of yet another embodiment of the facet implant of the present invention.

FIG. 7 is a side plan view of the of the facet implant of FIG. 6 when deployed.

FIG. 8 is a perspective view of another embodiment of the present invention with an introducer.

FIG. 9 is a perspective view of another embodiment of the present invention.

### DETAILED DESCRIPTION

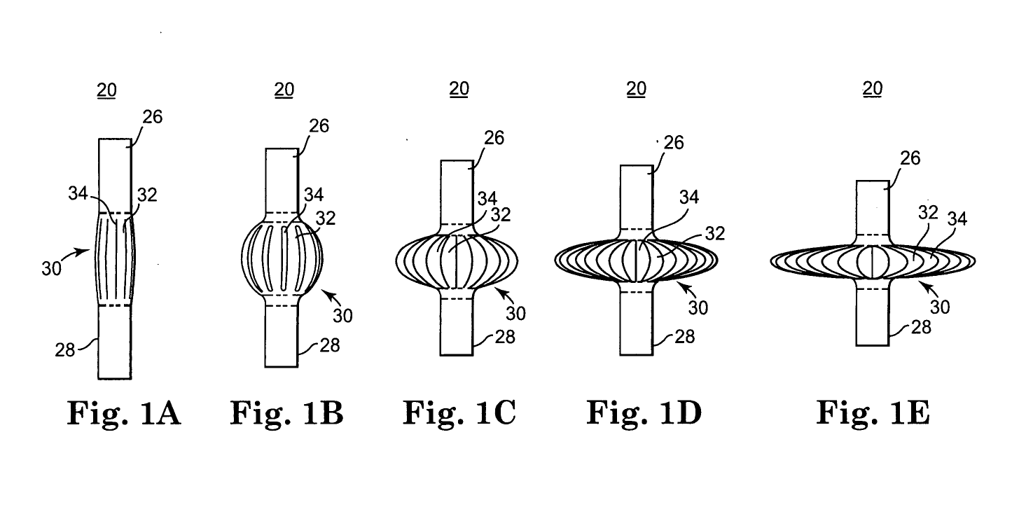

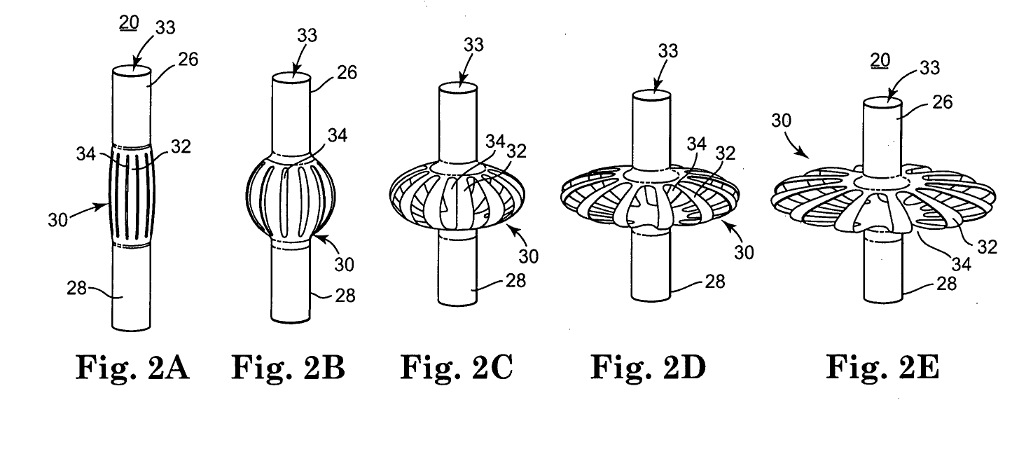

As illustrated in FIGS. 1-2, one embodiment of the present invention teaches an apparatus and method for stabilizing a facet joint 18 of a spine. One aspect of the present invention is the insertion of a facet implant 20 into one or both of a superior facet 22 and an inferior facet 24 through respective articulating facet surfaces 22A, 24A. In one embodiment the facet implant 20 is flexible and movably stabilizes the facet joint 18. The facet implant 20 may help to stabilize flexion, extension, rotation, and lateral bending of the spine and may also help to pillow or cushion the facet surfaces 22A and 24A by providing a new or supplemental articulating surface. After inserting the facet implant 20, the facet joint 18 may retain the full capability of the spine for movement. In further embodiments the facet implant 20 may be utilized to help fuse the facet joint 18 or to remove the cartilage between the facets 22 and 24. The facet implant 20 may be inserted and still preserve more than one, and up to all six, ranges of motion of the facet joint 18.

FIGS. 1A and 2A illustrate one embodiment of the present invention facet implant 20. The facet implant 20 includes a proximal member 26, a distal member 28 and an intermediate member 30. The proximal member 26 and distal member 28 may also be referred to as a lower and upper end, a first or second end, or a proximal and distal body, respectively. The “distal” and “proximal” ends of the facet implant 20 are only points of reference as the different ends of the facet implant 20 may be similar or the same. As illustrated, the distal member 28 and the proximal member 26 are the same length but in alternative embodiments may be selected as desired to conform to the implantation site.

Each of the proximal member 26, distal member 28 and intermediate member 30 may take the form of a substantially cylindrical structure having an interior passage 33. The interior passage 33 may be a substantially hollow interior lumen or axial bore along the longitudinal axis of the facet implant 20. In one embodiment the interior passage 33 runs contiguously through each of the proximal member 26, distal member 28 and intermediate member 30. In other embodiments, further discussed below, the interior passage 33 may only extend through a part of the longitudinal axis and may further include receiving areas, screw threads, or other devices that act as insertion aids or securing means.

The intermediate member 30 may include a number of strips 32 defined and separated by slits 34. When the proximal member 26 and distal member 28 are brought closer together the strips 32 may bulge out from the longitudinal axis of the facet implant 20. The strips 32 may also be described as distorting, enlarging, expanding, increasing, inflating, puffing, stretching, or widening relative to the longitudinal axis. The strips 32 may in many ways mimic the movement of the natural facet joint 18. FIGS. 1B-D and 2B-D illustrate the intermediate member 30 in a variety of stages between the first shape and the second shape during deployment. As may be appreciated, depending on the structure of the strips 32, the strips 32 may bulge in a variety of different formations. The bulging out of the strips 32 causes the intermediate member 30 to form a second shape. As illustrated the second shape is substantially in the form of a rounded disc. The second shape, however, may form a variety of shapes such as a flattened disc, a barrel, elliptical, parabolic, spherical, or other shapes.

As may be appreciated, the intermediate member 30 may be in a number of shapes after insertion depending on the size of the facet joint 18 and the separation of the superior facet 22 and an inferior facet 24. The insertion of the facet implant 20 into the facet joint 18 and placement of the strips 32 will be discussed further below.

The facet implant 20 may provide stabilized enhancement of the opposing facets 22 and 24 without immobilizing the facet joint 18. The intermediate member 30 may be adjusted to require a greater or lesser amount of force to bend, move or rotate by changing the properties of the strips 32 of the intermediate member 30, such as affecting stiffness, bendiness, stretchiness, hardness, etc. In further embodiments the way in which the strips 32 are woven, connected to each other or the rest of the facet implant, coated, or other features or characteristics may be adjusted. In this manner, the degree and type of stabilization of the facet joint 18 may be selectively adjusted. The facet implant 20 may also be utilized as a broach to abrade, rasp, scrape, file, or otherwise remove cartilage tissue from the facet joint 18 while preserving the capsular ligaments. This embodiment is further described below.

To bring the proximal member 26 and distal member 28 together during insertion, and to bulge out the strips 32, may require the exertion of a defined amount of compression force dependant on the characteristics of the strips 32. The strips 32 may have a variety of stiff or springy characteristics such that impart a desired force on the facet joint 18 after implantation. In one embodiment the bulging of the strips 32 may store a defined compression force. The compression force may be due to the strips 32 having a shape memory or some other bias towards one position. In such an embodiment, after implantation, the strips 32 provide a restoration force on the facet joint 18 such as to resist compression of the facet surfaces 22A and 24A towards each other. The facet implant 20 may therefore help to maintain separation of the facets 22 and 24 and facet surfaces 22A and 24A. In such an embodiment the strips 32 may act as a shock absorber between facets 22 and 24. The restoration force may also provide stabilization for translational and rotational movement. As may be appreciated, during the insertion of such an embodiment a greater amount of cartilage or other material from the facet joint 18 may need to be removed, excised, ablated, etc.

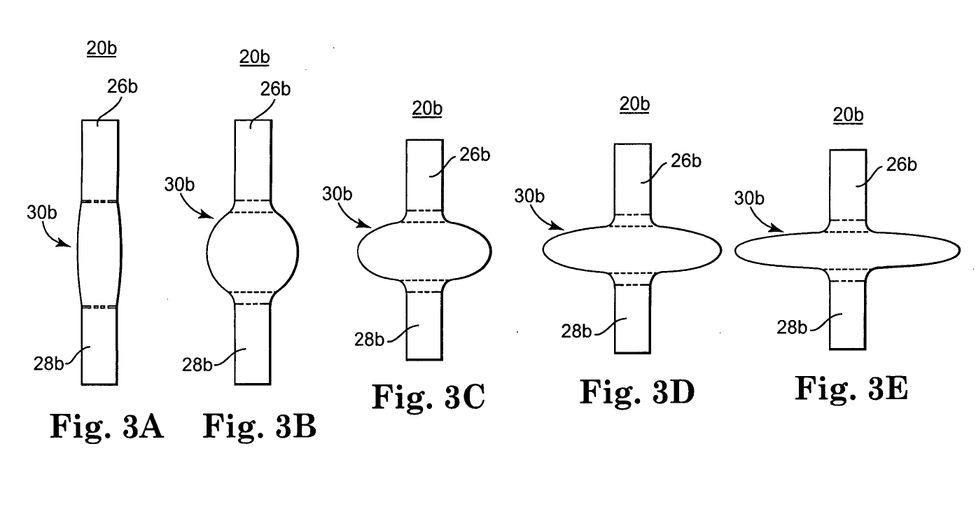

Such a restoration force may also be aided or otherwise moderated by a cushioning member inserted or placed into the facet implant 20, such as a small balloon filled with liquid. As illustrated in FIGS. 3A-E, a facet implant 20b with an intermediate member 30b including a balloon or other substantially solid structure may provide more cushion and a more complete articulating surface. In further embodiments the restoration force exerted by the strips 32 may be minimal or non-existent. In certain embodiments the strips 32 of the facet implant 20 may provide an articulating surface to the facet joint to prevent undesirable rubbing or grinding.

In other embodiments the shafts that form the proximal member 26 and distal member 28 may be substantially solid with the intermediate member 30 being hollow and forming the interior passage 33. In still other embodiments just the proximal member 26 and the intermediate member 30 may form the interior passage 33. In such embodiments, an insertion device may be inserted through the interior passage 33 of the proximal member 26 and intermediate member 30 and engage a receiving area of the distal member 28.

In addition, the facet implant 20 may include one or a number of other materials integrated therein to contribute other structural properties to facet implant 20. For example, harder materials, such as, but not limited to, stainless steel, titanium, nitinol and other materials or alloys may be added to the proximal member 26, distal member 28 or intermediate member 30 to vary the structural characteristics of the facet implant 20.

The distal member 28 and proximal member 26 of facet implant 20 may both include a smooth external surface. In further embodiments one or both of the proximal member 26 and distal member 28 may include a structure to aid in securing the facet implant 20 into the facet joint 18, such as, for example, a screw thread. Other types of structures may be utilized to enhance the fixing the facet implant 20 to the bone, such as hooks, spikes, traebecular metal material such as porous tantalum, etc.

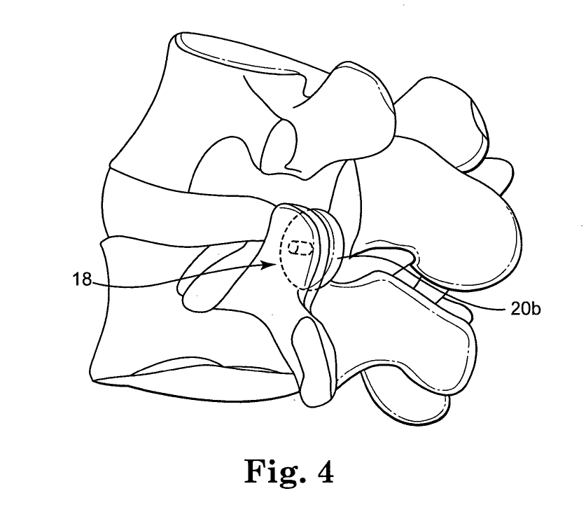

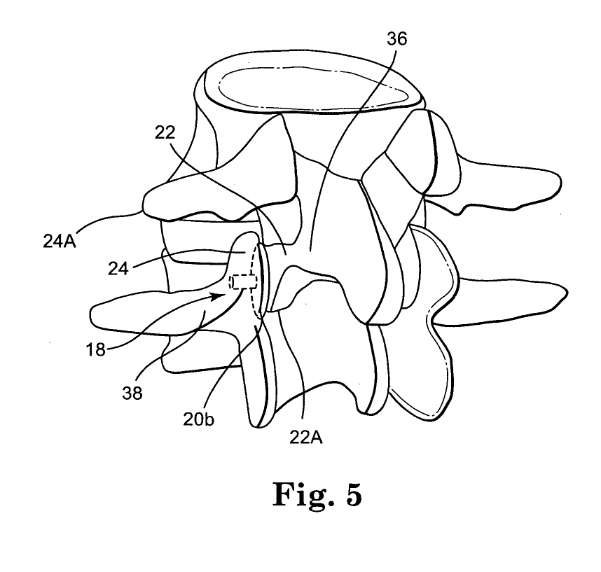

With reference to FIGS. 4 and 5, one method of insertion of the facet implant 20b will now be described. The facet implant 20b may be inserted so that the intermediate member 30b is disposed between the facets 22 and 24. The distal member 28b and the proximal member 26b may be secured through the facet surfaces and into the facets 22 and 24. In alternative embodiments, the distal member 28b may be secured into or through the superior or inferior pedicle depending on the angle of insertion.

During insertion, a facet joint 18 is first selected. A fluoroscopy or other internal image may be taken to help determine the insertion location and path. Selection of the depth and the angle at which the hole is made through the opposing facets 22 and 24 determines the positioning of the facet implant 20. The choice of location and direction for insertion of the facet implant 20b determine where the procedure is started.

A small incision may be first made at an appropriate location and the tissue retracted. Utilizing a drill or other boring instrument a hole may be created through each facet surface 22A and 24A. If desired, the hole may also be into or through a superior pedicle 36 and/or an inferior pedicle 38. The hole may be created to a desired depth and at a desired angle to receive the selected facet implant 20b. Taking a second fluoroscopic view may aid in the placement of the hole.

The facet implant 20b is then inserted into the hole. The facet implant 20b may be inserted with or without the aid of insertion tools. Some insertion tools that may be helpful may include a guidewire, a cannula, or other bone access instrumentation such as a surgical guideframe. The insertion of the facet implant may also be accomplished using minimally invasive techniques. Incorporation of such tools into the present method may be accomplished by one of ordinary skill in the art.

The distal member 28b of the facet implant 20b may ‘bottom out’ on the end of the hole in order to provide a stopping point for insertion of the facet implant 20b. In further embodiments, the hole may be deeper than required for placement of the facet implant 20b. In such cases the distal member 28b of the facet implant 20b may be secured in the hole at a desired depth by screws, adhesives, bone anchors, etc. Before insertion of the facet implant 20 a measuring device may be inserted into the hole to measure the depth of the hole.

The facet implant 20b may be positioned such that the when compressed along the longitudinal axis the intermediate member 30b deploys into the facet joint 18. The facet implant 20b may be then compressed such that the intermediate member 30b balloons out into the interior facet joint 18 space. The intermediate member 30b may bulge a desired amount into the space between the facets 22 and 24. The proximal member 26b may be then secured such that the interior member 30b remains in the desired portion.

In some embodiments ablation of the facets 22 and 24 and the surfaces thereof and/or the connective tissue and cartilage between facets 22 and 24 may be required or desired depend on each particular patient's indications. When using a facet implant 20b like shown in FIGS. 3A-E more of the facet joint 18 may need to be removed or compressed to allow room for the facet joint 18 to accept the facet implant 20b. The facet implant 20 may require less removal of tissue. Moreover, various distal members 28 and proximal members 26 of different sizes and lengths may be attached by a range of intermediate members 3o of various sizes to customize the facet implant 20 for each patient and each facet joint 18.

In still further embodiments the joint capsule may be removed and the facet joint 18 accessed via a posterior approach. The joint capsule may be cut open and the joint space retracted. The articulating surfaces could then be excised if desired and the implant placed into position.

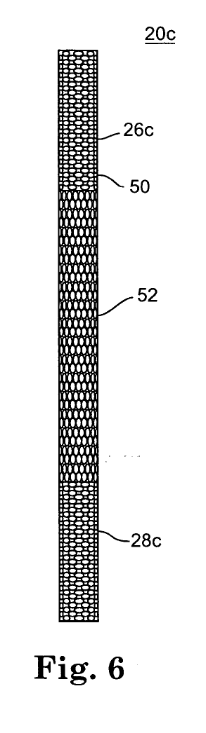

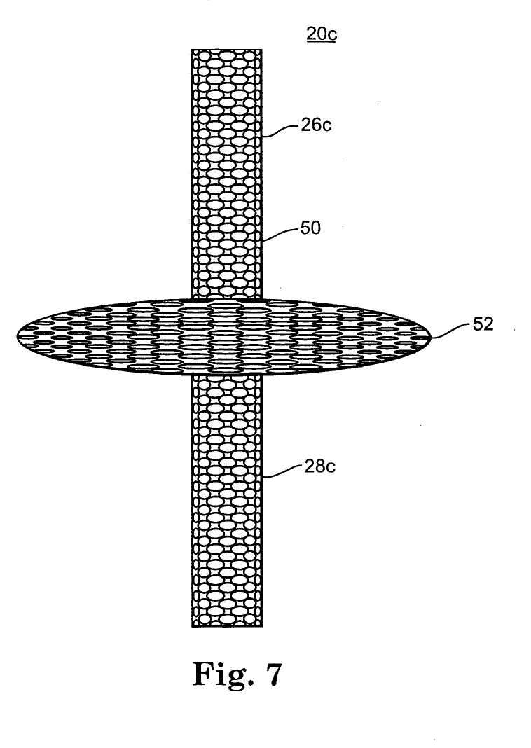

In another alternative embodiment illustrated in FIGS. 6-7 a facet implant 20b may be formed of a cord 50 or weave structure. The cord 50 may be inserted through the opposing facet surfaces 22 and 24 and affixed into the desired position in the manner previously described. Such a cord 50 may include a polyethylene terephthalate yam such as is utilized in the Dynesys™ system described further in EP 0669109 B1 and WO 94/17745, both of which are incorporated by reference for all that they teach and disclose. An intermediate section 52 of the material may be designed such that it bulges in a manner similar to the facet implant 20.

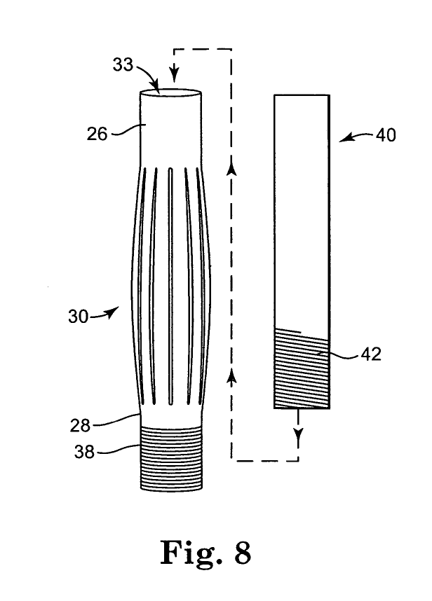

As illustrated in FIG. 8, the surface of the facet implant 20 may include screw threads 38 to aid in affixing the facet implant 20 in the desired position. The screw threads 38 may be formed on the distal member 28, proximal member 26 or both. In some embodiments the screw threads 38 may be integrally formed as part of the facet implant 20 and in other cases may be another structure joined to the facet implant 20. In one example the screw threads 38 are disposed on the exterior of the distal member 28, the proximal member 26 and the intermediate member 30. In other embodiments the screw threads 38 may only be on the distal member 28. In such embodiments the solid interior of the distal member 28 may include a receiving area that is engaged with a screwdriver or other insertion device. In effect, the screw threads 38 may pull the facet implant 20 into position. The proximal end 26 is then pushed into the hole to bulge out the strips 32 of the intermediate member 30 a desired amount and secured.

The interior passage 33 may further receive an introducer 40. The introducer 40 may be a removable member that extends through a portion of the interior passage 33 of the facet implant 20. The introducer 40 may include external left hand screw threads 42 to engage with corresponding internal threads (not shown) on the inside surface of some or all of the interior passage 33 of the facet implant. The introducer 40 provides structural stability to the intermediate member 30 to help prevent binding, bending, or other movement of the intermediate member 30 during placement. The left-hand screw threads 42 allow the introducer 40 to engage the screw threads on the interior passage 33 during insertion. After placement of the facet implant 20 into the desired position, the introducer may be removed from the interior passage 33. The intermediate member 30 is then deployed by moving the proximal member 26 closer to the distal member 28. The end of the proximal member 26 may be sealed using a set screw or plug, or, alternatively, left open.

As may be appreciated, the flexibility and support provided by the intermediate member 30 may be selected depending on the desired clinical outcome. In other words, the intermediate member 30 may be made more or less stiff, springy, or resistant to torsional force. The materials used to make the intermediate member 30 may contribute to the amount of “stiffness” provided by facet implant 20 to the facet joint 18.

In another alternative embodiment, the facet implant 20 may include a proximal member 26, intermediate member 30 and distal member 28 of varying widths or lengths. In such an embodiment the hole created in the facet surface through which the distal member 28 is placed may be larger or smaller in order to reduce the risk of fracturing the facet surfaces 22 and 24 during placement.

Furthermore, a facet implant 20c may be utilized to remove cartilage and other connective tissue from the facet joint 18. Such a facet implant 20c may have cutting or otherwise sharp edges along the strips 32c. When the facet implant 20c is inserted into positioned, rather than being secured the facet implant 20c is spun or otherwise moved in the facet joint 18. This will cut or otherwise excoriate the material within the circumference of the intermediate member 30c. The removed material may then be sucked out through the interior passage 33c of the facet implant 20c. The facet implant 20c may also be removed before removal of the material. In such an embodiment the distal member 26c may be kept shorter.

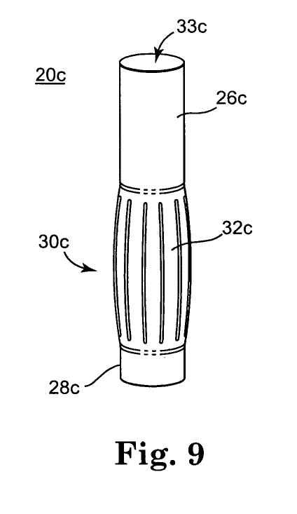

In the case where the facet implant 20c is utilized to abrade material, the facet implant 20c may not require a distal member 28c or may include a distal member 28c of minimal size. See FIG. 9. Such a facet implant 20c may not be secured into the facet joint 18 and so may be referred to instead as a facet tool rather than a facet implant. Once the facet tool removes the cartilage tissue facet tool may be inserted into the facet joint 18 to repair or replace the facet joint 18 or may be removed to allow another implant to be inserted. In further embodiments the facet tool may be filled with an in-situ curable material to fuse the facet joint 18.

In another alternative embodiment the facet implant 20 may be filled with an in situ curable polymer or other material to fuse the face joint.

Various modifications and additions may be made to the exemplary structures and steps discussed. Various combinations, permutations, and rearrangements of those structures and steps may similarly be made without departing from the scope of the present invention. Accordingly, the scope of the present invention is intended to embrace all such alternatives, modifications, and variations as fall within the scope of the claims, together with all equivalents thereof.

1. A facet implant for insertion into a facet joint comprising:
a distal member;
a proximal member; and
an intermediate member connecting the distal member and the proximal member, the intermediate member for positioning between a first facet surface and a second facet surface and transformable between a first shape and a second shape.

2. The facet implant of claim 1 wherein the distal member, proximal member, and intermediate member form a substantially cylindrical shape when the intermediate member is in the first shape.

3. The facet implant of claim 1 herein the distal member, proximal member and intermediate member include a lumen through a longitudinal axis.

4. The facet implant of claim 3 further comprising a removable introducer engaged through a portion of the lumen.

5. The facet implant of claim 1 wherein the second shape of the intermediate member is that of a rounded disc.

6. The facet implant of claim 1 wherein the intermediate member includes a number of slits running along a longitudinal axis between the distal member and the proximal member.

7. The facet implant of claim 6 wherein the slits form a number strips running between the distal member and the proximal member along the longitudinal axis, the strips bulging out when the intermediate member is in the second shape.

8. The facet implant of claim 1 wherein the slit further comprises a cutting surface.

9. The facet implant of claim 1 wherein the distal member further comprises screw threads disposed on an outer surface.

10. The facet implant of claim 1 wherein the facet implant is formed of a woven material.

11. The facet implant of claim 1, wherein the facet member is made of a biocompatible material and is one or more selected from the group consisting of metals, alloys and polymers.

12. A minimally invasive facet implant comprising:
a first implant member positionable through a facet of a first vertebra;
a second implant member positionable through a facet of a second vertebra, wherein the first vertebra facet opposes the second vertebra facet; and
an intermediate implant element connecting the first and second implant members, the intermediate implant element positionable between the first and second vertebra facets.

13. The facet implant of claim 1 further comprising a cushioning member disposed in the intermediate member.

14. A method of stabilizing a facet joint, comprising:
drilling a receiving hole through one or more of an opposing pair of facet surfaces; and
inserting a facet implant into the receiving hole, the facet implant including an intermediate element positioned between the opposing facet surfaces wherein the intermediate element is transformable between a first shape and a second shape.

15. The method of claim 14 further comprising the facet implant providing a restoring force to the facet joint such that a desired spacing between the facets of the first and second vertebra is maintained.

16. The method of claim 14 further comprising providing an articulating surface.

17. The method of claim 14 further comprising removing cartilage from the facet joint before inserting the facet implant.

18. The method of claim 14 wherein inserting the facet implant further comprises screwing the facet implant into position.

19. The method of claim 14 further comprising inserting a cushioning member into the intermediate member.

20. The method of claim 14 further comprising rotating the inserted facet implant to cut away cartilage in the facet joint.

21. A method of preparing a facet joint for fusion comprising:
drilling a receiving hole through a first facet surface;
inserting a facet tool into the receiving hole, the facet tool including a number of strips positioned between the opposing facet surfaces wherein the strips are transformable between a first shape and a second shape and wherein when the strips are in the second shape the strips expose a cutting surface; and
moving the facet tool such that the cutting strips abrade a desired amount of cartilage from the facet joint.

22. The method of claim 21 further comprising removing the abraded cartilage from the facet joint.

23. A minimally invasive facet tool for abrading cartilage from a facet joint comprising:
a first tool member; and
a second tool member distally extending from the first tool member, the second tool member positionable at a position in the facet joint and including at least one edge for abrading the facet joint.
